(19) United States
(12) Patent Application Publication
(10) Pub. No.: US 2013/0123774 A1
(43) Pub. Date: May 16, 2013

(54) SURGICAL TIPS FOR PIEZOELECTRIC BONE SURGERY

(71) Applicant: Homayoun H. Zadeh, Calabasas (CA)

(72) Inventor: Homayoun H. Zadeh, Calabasas (CA)

(21) Appl. No.: 13/675,428

(22) Filed: Nov. 13, 2012

Related U.S. Application Data
(60) Provisional application No. 61/558,404, filed on Nov. 10, 2011.

(51) Int. Cl. A61B 17/16; A61B 18/14

(52) U.S. Cl. CPC A61B 17/16; A61B 18/14
USPC 606/39

(57) ABSTRACT

A series of tips for use with an ultrasonic or piezoelectric dental surgical device dental are used in osteotomy, ostectomy and osteoplasty procedures or any procedure requiring removal or shaping of bone or other hard tissues. In some embodiments, fissures are provided in a cutting end of the tip to facilitate osteotomy. The tips are shaped so that they are comfortable for the surgeon to use in a proper position, so that when a handpiece to which the tips are releasably attached is held in the conventional manner, the geometry of the osteotomy will be precise and desirable. When energized, the tips readily cut through bone or facilitate shaping of skeletal structures at the surgical site. Methods for use of the tips and systems in which the tips provided the cutting function are also described.

### FIELD OF THE ART

The present invention is in the field of surgical devices used for osteotomy, osteoplasty and ostectomy, specifically tips used with a piezoelectric surgical system used in dental surgery.

### BACKGROUND

Bone surgery operations that involve cutting as modeling of bone tissue (osteotomy), (ostectomy) and (osteoplasty) are notoriously difficult and require both precision and application of large mechanical forces to change the shape of mineralized bone or to remove bone tissue.

Traditionally, manual chisels and other hand instrumentation, as well as, rotary drills, oscillating and reciprocating saws are used to perform ostetomy, ostectomy and osteoplasty. Manual instruments have proven to produce unsatisfactory results often in part because bone has the tendency to shatter or split in unpredictable ways. The action of saws is limited to straight cuts with limited application. Saws also can produce excessive vibration and are difficult to handle and are non-selective in cutting into various hard and soft tissue structures. Drilling has two drawbacks, namely 1) the heat generated by drilling can inhibit bone growth and 2) the vibration of the drill can produce inaccurately shaped osteotomy and often damage the bone.

These difficulties led to the development of piezoelectric surgical devices. With these devices, high frequency vibrational energy produce a cavitation effect, which is preferentially exerted on hard tissues, minimizing trauma to surrounding soft tissue. The device typically has a handpiece with replaceable, selectable tips that are interchanged as a function of the procedure. The cutting action on the bone tissue is produced by variable modulation ultrasonic vibrations that are activated only on the cutting end of the tip. Ideally, the tip is the only tool that comes into contact with the mineralized bone tissue and the tip provides extremely rapid vibration and the necessary force and energy to cut bone.

Consequently, the energy applied to the bone tissue surface is highly directed and the affected area can be limited by the design of the tip. This feature allows the surgeon to perform an osteotomy or other procedure on the bone tissue with application of less mechanical force. This in turn lessens the trauma suffered by the bone tissue and the surrounding soft tissue to that caused by the friction of the cutting instrument and a small amount of heat that is absorbed into the bone. The vibrating tip is also less likely to damage the surrounding soft tissue because the energy caused by the vibrations of the tip is dissipated in the form of minor, localized heat and causes no irreparable damage.

While the development of the piezoelectric systems is an advantage over the older rotary technique, the highly focused application of vibrational energy emphasizes the value of improved tip designs. The tips should provide the ability to maximally focus the vibrational energy of the device while providing ease of use and comfort for the surgeon. Many existing tip designs do not always work well for their intended purpose. The current tips designed for osteotomy are designed with serrations at the tip, which leads to the production of an irregular osteotomy. The action of the surgeon to produce the cutting effect requires rotation of the handpiece, which requires wrist and larger muscle groups to control the osteotomy, leading to imprecise osteotomy design. Also, some osteotomy tips have abrasive walls that rely on diamond coatings to provide the abrasiveness, thereby creating excessive heat.

Accordingly, a need exists for piezoelectric surgical tips that are specially designed to produce precise cutting of hard tissues, that enhance the surgeon's ability to control the vibrational energy applied to the tip, and that enhance the surgeons control of the instrument. There is also a need for ultrasonic tips that reduce thermal injury to bone.

### SUMMARY OF INVENTION

The present invention is surgical devices systems, and methods for bone surgery. Specifically, the devises are tips used in piezoelectric surgery wherein the tip designs are specially suited for dental osteotomies and bone shaping. These devices are suitable, for any surgical procedures requiring osteotomy, ostectomy, osteoplasty of hard tissues, where a great deal of precision is required, while avoiding excess thermal tissue injury. Such surgical procedures, may include harvesting of bone from donor sites, extraction of impacted or erupted teeth, preparation of osteotomy to place implants and other anchorage devices, corticotomy to facilitate orthodontic tooth movement, endodontic procedures, osteotomy to gain entrance into sinus lumen, osteotomy in orthognathic surgery, otorhinolaryngology surgery, orthopedic surgery to cut or shape various bones, and neurological surgery to operate on bone in close proximity to neurovascular structures.

Accordingly the tips of the present invention are designed and structured to provide a highly accurate, precise, and controlled applications of high frequency vibration energy when the system is energized and enable the surgeon to readily cut, shape and otherwise mold bone and other hard tissues.

The tips of the present invention are designed to be used with existing piezoelectric surgical apparatus such as Piezosurgery®, Piezotome®, PiezAart®, Variosurg®, Piezon®, SurgyStar®, Ultrasonic Bone Surgery® (UBS), Synthes®, and INTRAsurg®. These systems typically allow ready exchange of different tip designs depending on the procedures and the unique requirements of a patient's individual skeletal structure. In use, the tips are attached to a handheld instrument via a base at a proximal end such that the tip that is releasable from the instrument and terminating in a tip at the proximal end such that vibrational energy from the system, usually under manual control of the surgeon, is applied to the surgical site.

The design and structure of these enhanced tips provide enhanced and unique cutting and modeling capabilities such that the overall feel of the instrument is comfortable for the surgeon to use, and such that the tip can be readily be placed in the proper position to perform a procedure when the system is actuated and the energy of the system is activated to energize the handpiece and the tip. These tips are suitable for use with existing piezoelectric surgical systems and require no modification to the design or control elements of such systems.

### DESCRIPTION OF THE FIGURES

FIGS. 1A-1F are a fissured osteotomy tip having fissures or serrations along the length of the diotal end of the tip.

FIG. 2 is an abrasive trumpet tip having an abrasive surface along the conical exterior at the tip.

FIG. 3 is an indented periotome tip having concavities along the tip to reduce contact and facilitate passage of irrigation solution.

FIG. 4 is an indented saw tip having concavities along the teeth of the saw tips.

FIG. 5 is a fissured osteotome having features in common with the embodiment of FIG. 1 but with specified indentations along the length of the distal end of the osteotome.

### DETAILED DESCRIPTION OF INVENTION

The surgical system for bone surgery according to the invention provides a handpiece comprising a tip capable of operating on bone tissue. For this purpose, according to selected embodiment described below, various devices in addition to the tips can be mounted on a suitable handpiece. The handpiece has to provide for external and/or internal irrigation to the tip. Additionally, lighting may be provided for enhanced visualization.

The surgical system may also have a controller console with dedicated software to control the electrical acuity of the system and the selected application of vibrational energy. Optionally, the console controller has a touch pad or key pad or foot pedal for operator input and control.

The control electronics allow the operator to control the application at vibrational energy including the modulation between low frequency and high frequency bursts. In this manner the user controls the vibrational energy that is ultimately transmitted to the tip of the handpiece.

Various types of piezoelectric handpieces are utilized for dental surgical applications. A typical ultrasonic handpiece uses a standard tip with an inner aspiration fluid flow passage and has uniform inner and outer diameters along its length. Typically, such handpieces use some type of vibrating piezoelectric transducer, which converts electrical energy into mechanical energy. The mechanical energy is used to vibrate a tip, or needle, of the handpiece and the tip distal end emulsifies the tissue with which it comes into contact, a process referred to as cavitation. The tip is preferably configured to attach to the handpiece, such that a hollow interior of the tip mates to a fluid channel on the handpiece, to provide a passage of irrigation fluid from an external source, through the handpiece via the channel and to the distal end of the tip to reduce the temperature of the tip. Preferably a fluid-sealed fixture mounted on the distal end of the handpiee or the proximal end of the tip, or a mating fixture or both, seals the fluid path between the handpiece and the tip.

The handpiece may comprise a piezoelectric transducer that generates vibrational energy that is transmitted to the tip. The tip is made to vibrate at selected frequencies of between approximately 22 to 29 KHz to make an extremely fine and precise cut in bone tissue.

As can be seen in the following description and figures, these tips have structural features that are configured to selectively supply or transmit vibrational energy to bone, variations in the orientation and shape of the tips allow the surgeon to select the specific tool necessary for the particular procedure dictated by the patient and the desired outcome. In each case, the overall efficiency of the handpiece is improved by the design and selective placement of structural features of the tip including specifically the orientation of serrations at the distal end of the tip along a region immediately distal to the point of the tip as described below.

The tips of the present invention are installed on an ultrasonic piezoelectric handpiece and energized with ultrasonic energy to vibrate and resonate such that the vibrating tip is brought into working contact with hard tissues such as bone or tooth structure. When contact is made and energy applied, the tip will abrade the hard tissues in contact with the working end of the tip such that the hard tissues can be removed in a controlled fashion. Even more specifically, tips are useful for preparation of jawbones to receive endosseous implants.

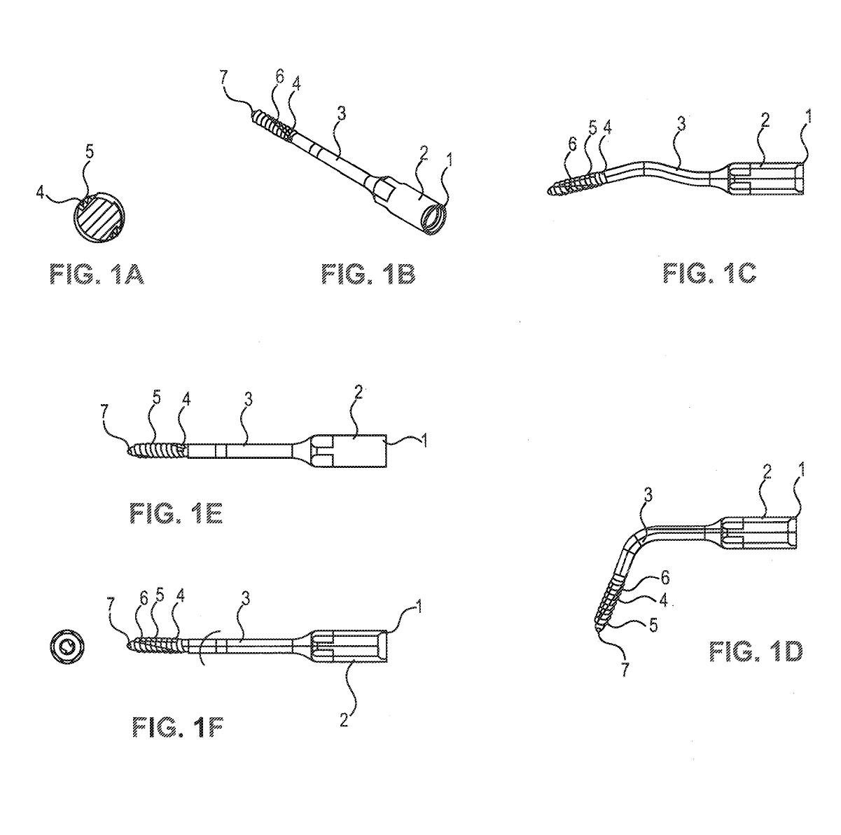

FIGS. 1A-1F are a fissured osteotome or osteotomy tips having structures along the length of the tip to enhance the ability to cut or model bone. These tips also have unique geometries that aid the visibility and hand control by the surgeon during a bone removal or modeling procedure.

A surgical device for bone surgery according to the invention is described with the aid of the accompanying figures. As shown in FIGS. 1A-1F, the surgical device is comprised of a tip having a base 1 and a body 2. The base 1 is adapted to releasably engage a handpiece (not shown) that is operably connected to a piezoelectric surgical device operated by a surgeon by means of a controller. Typically, the controller allows the surgeon to selectively apply vibrational energy, through the handpiece, to the box 1 of the device, through the body 2 of the device. The most proximal portion of the body 2 forms the base 1 and closely conforms and preferably releasably attaches to the handpiece so that a piezoelectric or ultrasonic transducer associated with the device transmits vibrational energy through the body without unacceptable loss to the shaft 3 for transmission to the distal end. The shape and design of the base 1 and the body 2 are only constrained by their ability to reliably transmit vibrational energy to the operative portion of the tip. Typically, the body 2 tapers into an elongated shaft 3 that terminates in the “cutting end” of the device.

The shaft 3 may take a variety of angles or conformations to allow the advantageous orientation of the distal end of the tip relatively to the handheld piece. The overall design, curvature, and length of the elongated shaft 3 may vary according to the position in which a cutting is desired in the procedure. The size and diameter of shaft 3 is also variable according to the practical constraints described herein. The most common diameters range from 0.5 millimeters to 5.0 millimeters, the most common lengths range from 2 millimeters to 15 millimeters in the overall tip. Thus, the portion of the device devoted to each of the base, body, base 1, body 2, an elongated shaft 3 are only constrained by the operative ability to transmit vibrational energy to the distal end and the need to have the distal most end designed as described herein.

The most distal end of the tip has a shaft with a length thread that is generally described as a cutting end and may be disposed at variable angles relative to and along the elongated shaft, but typically deflects at an angle ranging between 0 and 90 degrees. Within the cutting end, the structures that directly contact the bone to transmit energy are the cutting surface. Various structural means for transmitting the energy are formed to create the cutting surface. The formation of a working cutting surface along the length of the distal end of the shaft 3 to form an operative cutting end is an important feature of the invention and difference from other known tip designs. Known tips tend to have the cutting surface located only at the most distal end of the tip such that the surgeon must continuously rotate or re-position the tool to perform an osteotomy having more than a minimally defined linear length. Moreover, the present design allows the surgeon to position the distal end of the tip between two structures such that the vibrational energy is transmitted along the entire length of the cutting portion of the tip.

In the embodiment of FIGS. 1A-1 F, the overall cutting end is generally comprised of the shaft length and a number of fissures 4 that can take a variety of different geometries to form the cutting surface. The fissures 4 have characteristic sharpness provided by an edge 5 that is sharpened along one or more of the edges 5 that pass around the circumference of the shaft 3 to provide a series of circumferential edges 5 that may be located along a continuous fissure 4 or may be located at an edge 5 at each individual fissure 4. The fissures 4 with edges 5 are preferably circumferentially arranged around the entire external surface of the distal end of the shaft 3 to form the cutting tip, but may be limited to only the most distal portion depending on the design of the individual tip.

The geometry of the fissure 4, the edge 5 and the overall cutting surface may include cylindrical, common-tapered cylindrical, flame-shaped, oblong, ovoid, or spherical. The orientation of the fissures 4 are generally in a cylindrical or spiral shape around the cutting end and may terminate at a distal point 7 that is smaller in diameter than each individual fissure 4. The distal point 2 and a length of the distal end comprising the cutting end may also have formed therein an indented channel 6 that may be linear or may form a partial or complete spiral along with the orientation of the fissures 4 to allow irrigation or other passage of fluid and materials along the cutting end.

The indented channel 6 may be replaced or supplemented by an internal channel (not shown) that runs the length of the distal end of the cutting end, preferably from an opening proximate to the distal point 7 to a fixture in the base 1 (traversing the elongated shaft 3) such that irrigation fluids or aspirated materials may travel in either direction along the path of the shaft 3. As will be apparent, the internal irrigation/aspiration channel or indented channel 6 may be formed independently in any tip disclosed herein. Referring specifically to FIG. 1A, a head on view of the tip of the invention shows the orientation of the edge 5 relative to the distal point 7 of the tip. Advantageously, this configuration minimizes the surface area at the point of contact between the cutting surface of the tip and surrounding bone tissue, when the length of the cutting surface of the tip engages skeletal bone structure during the application of high frequency vibrational energy. The number of individual turns in the fissures 4 and their pitch can vary but the number of turns comprising a cutting end it is generally between 2 and 20. Modulating the pitch and the the dimensions of the fissures will allow the tips to have varying degrees of coarseness suited for varying bone densities. Referring specifically to FIGS. 1E and 1F, the dimensions of the cutting end at the tip are in overall size and orientation not significantly different from existing tips. Typically, the small length of the device is less than 50 mm. and may be approximately 36.7 mm. The diameter of the base 1 is approximately 3.7 mm and has an overall length, when taken together with the body 2 is 10.3 mm. The body 2 taper (3.0 mm) to the elongated shaft 3 having an overall length of approximately 23.4 mm. The cutting surface is typically found at the distal-most end (approximately 15 mm) and wherever the fissures 4 and edges 5 may be formed in the most distal 10 mm or less. In the spiral design, the pitch; i.e. the distance between 2 adjacent edges 5 at an identic points A-A along the length of the cutting surface may be 1.0 mm.

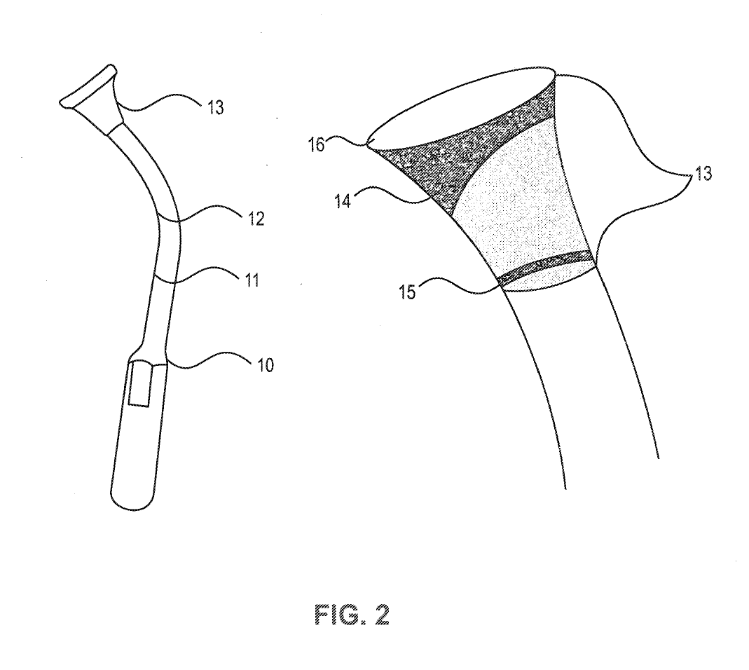

Referring to FIG. 2, an embodiment of the invention is termed an abrasive trumpet and has a tip comprised of cutting surface formed by an abrasive coating formed in a trumpet or flare-shaped distal end of the tip. Referring to FIG. 2, the tip has a base and a body 10 that serve the purpose of releasable attachment to a handheld device and transmitting vibrational energy as described above. An elongated shaft 11 may be of any length and orientation to position the distal end of the tip in operative configuration for a surgical procedure. The elongated shaft 11 may have a preformed curve 12 that performs the same function. As with the embodiments described in FIGS. 1A-1F above, the overall measured angle along the base, through the elongated shaft and the preformed curve 12 generally create an angle between 0 and 180 degrees and most preferably between 0 and 90 degrees.

In the embodiment of FIG. 2, an abrasive coating 14 at the distal most portion of the tip provides a cutting surface that can comprise all or a substantial portion of the most distal end. The distal point (reference 7 in FIGS. 1B, 1E, 1D and 1F) is formed into an annular and most distal end of the tip. As in the embodiment of FIGS. 1A-1F, fissures, channels, or additional edges may be incorporated into the distal most tip by conventional manufacturing methods. The abrasive coating 14 of the distal end may be found by forming an additional lay of an abrasive coating material at a selected portion of the tip to yield the cutting end. The preferred method to create the abrasive surface is diamond coating, see U.S. Pat. No. 5,299,937 and Sein et al., Diamond and Related Materials, Vol. 11:3-6, pp. 231-35 (2002). Each of chemical etching, laser etching, EDM manufacturing and coating of a distal end with a diamond slurry are all conventional methods for forming an abrasive coating 14 at a selected region of the tip to form the cutting surface. The abrasive coating 14 may cover an entire portion of the distal end or maybe selectively be formed in any shape or format as desired. For certain surgical procedures, the distal end is ideally formed into a trumpet of flare shape 13 with the smallest circumference 15 adjoining the elongated shaft 11 and having a circumference substantially identical thereto. The elongated shaft 11 can have variable diameters and lengths, however the most common diameter will range from 2 millimeters to 5.0 millimeters at the distal most tip and common lengths of the entire cutting tip range from 2 millimeters to 8.0 millimeters.

Although a flare or trumpet shape is shown in FIG. 2, essentially any tip can be provided with a selectively placed abrasive coating 14 to enhance the bone cutting or bone modeling function as illustrated by this embodiment. In use, the embodiment of FIG. 2 is primarily used for reshaping bone along a perimeter of a lateral window made during maxillary sinus surgery. The circular end portion 16 of the trumpet shaped tip 13 may be solid and smooth, solid and covered with abrasive material and maybe concave, flat, or convex but is preferably substantially flat along the end surface.

The bone modeling function is best provided by a distal end that is substantially flat and smooth so that the distal end can be placed against bone that is not desired to be cut or modeled or against soft tissue such that the cutting function provided by the translation of vibrational energy does not extend through the distal most portion of the tip. For cetain applications, the terminal end may consist of a flat surface. In other designs, the terminal end may be hollow. The hollow end may be contiguous with the irrigation channel to allow irrigating fluid to exit from the end, producing hydrolic pressure, which may be advantageous to simultaneously dissect soft tissues away from the tip.

As with the embodiments described above, channels or grooves may be formed in any exterior or interior surface of the tip, including traversing the elongated shaft 11 to provide for irrigation or aspiration of materials.

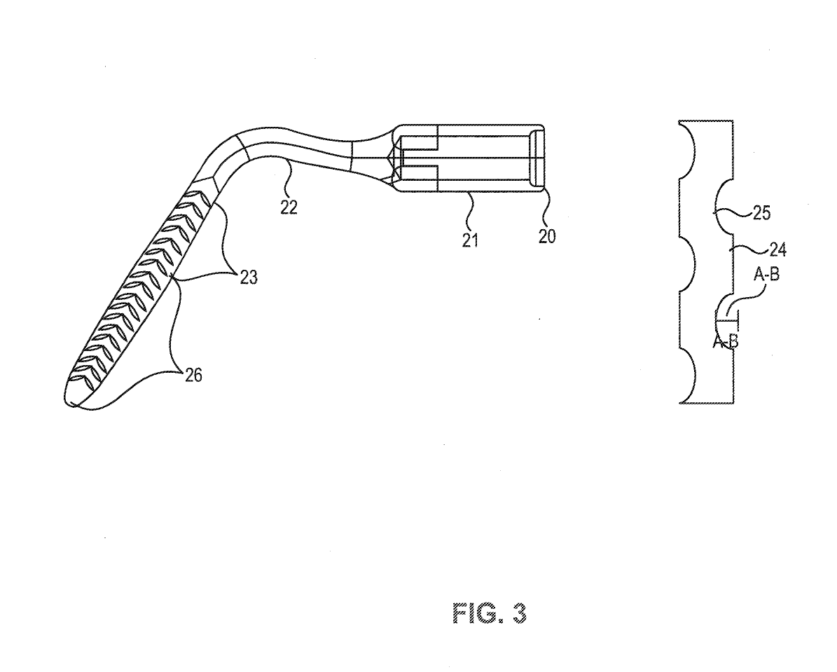

Referring now to FIG. 3, an indented periotome tip is shown wherein indentations 25 formed along the distal most portion of the tip substantially reduce the contact area between the cutting end of the tip at the distal-most end and the surrounding soft tissue. This configuration allows irrigating solution to freely enter the indentations 25 spaces between indentations 25 and along the distal end of the tip and the soft tissue. The reduction in the surface area and friction along the length 23 of the cutting surface also reduces generated heat and promotes dispersion of heat between the periotome and the bone. As in the above described embodiments, the device has a base 20 designed to releasably engage a handheld device as part of the piezoelectric surgical system and has a body 21 that tapers into an elongated shaft 22 that may be configured in any angle (as described above) to facilitate performing the surgical procedure.

The indented periotome may terminate in a distal point 26 that is smaller in diameter than the length 23 of the cutting surface of the tip containing the indentations 25 along the length thereof. The portion of the distal end of the tip containing the indentations typically has spaced apart flat surfaces 24 that are substantially equivalent diameter of the tip and has concavities 25 formed or cut along the length.

As shown in FIG. 3, an alternating spacing between the concavities 25 and the flat outer surfaces 24 facilitate the free passage of fluid along the length of the tip. The distal end of the indented periotome tip may be cylindrical or may be oval in cross section to eliminate the overall cross section of the device, for example to allow insertion of the periotome inside the periodontal ligament space, between a tooth and the surrounding bone structure.

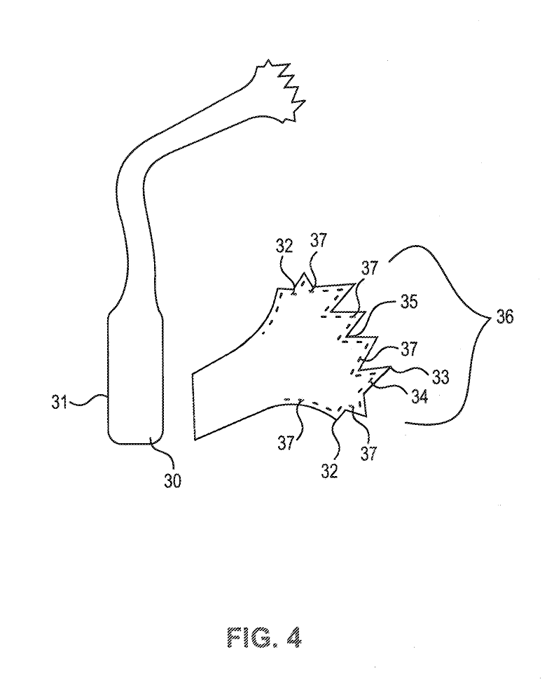

Referring to FIG. 4, an indented saw tip is provided for linear cutting of bone tissue along the length of a saw-tipped edge 36. In this embodiment, each indented saw tooth 34 has a series of indentations or concavities 37 along the edges of each individual tooth 34. The concavities 37 preferably span each edge of each tooth 34 extending away from the point 33 along the lateral edge 32 of each tooth 34. The concavities 37 preferably also extend away on the lateral edge 32 adjacent to the last tooth at either end. The concavities 37 in the lateral edge 32 and the individual tooth 34 provide the free passage of solution around the teeth 34 and reduce the contact area between the saw teeth 34 and the surrounding bone and soft tissue.

As with other embodiments described herein, the reduction in the surface area contacted between the tip and the bone and surrounding tissue reduces friction and provides for more rapid dissipation of heat during bone cutting. As with the above described embodiments, the indented saw tip preferably has a base 30 and a body 31 designed for releasable attachment to a handheld device for transmitting vibrational energy to the distal most portion of the saw tip. The indentations or concavities 37 are preferably arranged in alternating positions on either side of the blade and can be semicircular, ovoid, or any shape that reduces the overall surface area at the point of contact between the tip and the surrounding tissue.

As described above, the concavities may be formed by known manufacturing methods including EDM, laser etching, chemical etching, mechanical etching or formation of grooves by any known technique. The size of the individual concavities may be adjusted to minimize the surface area between the contact of the tissue and the saw tip, while maintaining the structural integrity of the entire cutting end of the saw tip as a function of the desired size of the saw teeth 34 and the materials used to form the tip.

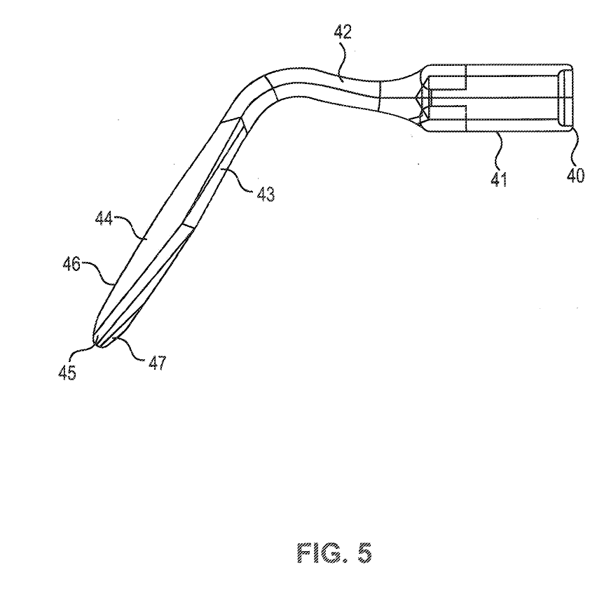

Referring to FIG. 5, a fissured osteotomy tip has a base 40 and a body 41 for releasable attachment to a handheld device as described above. An elongate shaft 42 is curved to bring the distal end of the tip into a desired configuration for applying vibrational energy to a surgical sight. As in the embodiment of FIGS. 1A-1F, a distal most end contains a fissured tip having fissures 44 and edges 46 that facilitate an osteotomy by cutting through bone upon the application of vibrational energy. The osteotomy tip has a groove or channel 46 that may traverse the cutting surface and be disposed in any portion of the length of the cutting end of the device to permit passage of irrigation or aspiration materials along the length of the tip. In this embodiment, the most distal point 47 is not tapered down to a point but remains essentially the same diameter as the length of the cutting end of the tip.

The method of the present invention is placing an osteotomy tip at a surgical site, activating a piezoelectric source to deliver high frequency energy to the site, removing one at the site along a length of the osteotomy tip at a cutting and wherein the cutting end is defined by an osteotomy tip having an indented or abrasive surface at the distal end thereof. The removal of bone may be a straight, linear excision or a shaping of bone structure at the points of contact with the osteotomy tip. The indented surface preferably provides a series of concavities along a length of the osteotomy tip to increase the active length of the cutting surface while decreasing the surface area of the immediate contact between the bone and the tip.

While the present invention has been particularly shown and described with respect to certain preferred and illustrative embodiments, it will be understood by those skilled in the art that variations and modifications may be made therein without departing from the spirit and scope of the present invention.

I claim:

1. A piezoelectric surgical apparatus comprising:
a handpiece comprising a tip having a base that releasably engages the handpiece, and wherein the tip has a cutting surface formed between a distal point of the tip and proximally along a length of the shaft to form a cutting surface along a length thereof; and
a source of vibrational energy for transmitting piezoelectric energy to the cutting surface of the tip.

2. The apparatus of claim 1, wherein the tip has a fixture at the proximal end of the base for sealing the base to the handpiece.

3. The apparatus of claim 1, wherein the tip is further comprises an internal fluid communication pathway.

4. The apparatus of claim 3, wherein the base of the tip and the handpiece have a mating fixture for sealing the fluid communication pathway.

5. The apparatus of claim 1 further comprising:
a controller having software to selectively apply piezoelectric energy through the handpiece to the tip.

6. The apparatus of claim 1, wherein the shaft has an indentation running distally from an area proximate to the distal point of the tip along the cutting surface of the shaft.

7. The apparatus of claim 1, wherein the shaft has a plurality of fissures formed in the cutting surface of the shaft.

8. The apparatus of claim 1, wherein the tip is comprised of a series of circumferential edges along the cutting surface.

9. The apparatus of claim 1, further comprising a lighting source to direct light at the distal end of the tip.

10. The apparatus of claim 1, wherein the source of piezoelectric energy is a transducer.

11. The apparatus of claim 1 wherein the vibrational energy is between 22 and 29 KHz.

12. A method for piezoelectric bone surgery comprising:
releasably attaching a piezoelectric tip to a handpiece,
orienting the handpiece to cause an elongated shaft at a distal end of the tip to contact bone along the length of a cutting surface of the tip, and
applying piezoelectric energy along the length of the cutting surface to remove bone.

13. The method of claim 12, further comprising applying piezoelectric energy through a point of the tip at the distal end of the shaft.

14. The method of claim 12, wherein the step of applying piezoelectric energy is comprised of contacting bone with a plurality of edges circumferentially surrounding the shaft and activating a source of piezoelectric energy.

15. The method of claim 12, further comprising the step of removing fluid through a channel running substantially the length of the cutting surface of the tip.

16. The method of claim 12, wherein the step of applying piezoelectric energy is comprised of activating a controller that selectively applies the piezoelectric energy through the handpiece.

17. The method of claims 12 further comprising the step of passing irrigation fluid through a fluid communication pathway internal to a body of the tip and to an opening proximate to the distal point of the tip.

18. The method of claim 12 further comprising activating a light source to direct light to the cutting surface of the tip.
